(19) United States
(12) Patent Application Publication
(10) Pub. No.: US 2026/0224469 A1
(43) Pub. Date: Aug. 6, 2026

(54) ALCOHOLIC EXTRACT OF STEMS OF JASMINE JASMINUM GRANDIFLORUM L., METHOD FOR OBTAINING SAME, AND COSMETIC COMPOSITION CONTAINING SAME

(71) Applicant: CHANEL PARFUMS BEAUTE, NEUILLY SUR SEINE (FR)

(72) Inventors: David LEGANGNEUX, Pantin (FR); Annie LERISSON, Pantin (FR)

(73) Assignee: CHANEL PARFUMS BEAUTE, NEUILLY SUR SEINE (FR)

(21) Appl. No.: 19/138,808

(22) PCT Filed: Dec. 15, 2023
(86) PCT No.: PCT/EP2023/086036

§ 371 Date: Jun. 13, 2025

(30) Foreign Application Priority Data
Dec. 16, 2022 (EP) 22306914.7

(51) Int. Cl. A61K 8/9789; A61Q 17/00; A61Q 19/00

(52) U.S. Cl. CPC A61K 8/9789; A61Q 17/00; A61Q 19/00; A61K 2800/805

(57) ABSTRACT

An alcoholic extract of jasmine, Jasminum grandiflorum L., stems, which includes at least oleuropein, isoquercitrin and ursolic acid, and the process obtaining the extract from jasmine, Jasminum grandiflorum L., stems. Also, a cosmetic composition including the extract and cosmetic uses of protecting the skin with the extract and compositions thereof.

### FIELD

The invention relates to an extract of jasmine, Jasminum grandiflorum L., stems, to the process of obtaining it, to a cosmetic composition containing it, and also to various cosmetic uses.

### BACKGROUND

Today, pollution, in particular in urban environments, has become a scourge to health and affects, firstly, the skin, the part most exposed to pollutants.

Pollution is a contamination of the interior or exterior environment by any chemical, physical or biological agent. Pollution of the air is composed of two main types of primary pollutants: particulate matter (PM), commonly referred to as fine particles (PM 2.5 μm, PM 10 μm), and gases (O₃, CO₂, CO, SO₂, NO₂) or volatile organic compounds. The small particles are generally produced by combustion and the larger particles by mechanical processes, which create, then suspend, the dust particles in the wind.

Due to its critical location, the skin is a major interface between the body and the environment and constitutes a biological barrier against a range of chemical and physical environmental factors.

The skin is a protective casing, preventing the penetration of substances, the size of which is less than a value of between 20 nm and 200 nm. The surface layers of the skin form a physical, chemical and immunological barrier against the entry of harmful environmental substances, including ambient particles, and also pathogenic agents and allergens. This barrier being intact, the pollutants (mainly ozone) cannot penetrate into the skin, thus preventing disruption of the biological mechanisms related to inflammation, to the immune response, to the evaporation of water and to the degradation of the extracellular matrix.

The effects of pollution on the skin can be manifested in different ways, among which may be mentioned:
appearance of wrinkles and fine lines, of pigment spots,
dull complexion,
dehydration of the skin, sebum excess,
acne,
irritation on the skin.

For this reason, there exists a need to provide a multifunctional active agent capable of effectively combating the harmful effects of pollution and of preserving and restoring the cutaneous barrier function in order to maintain an optimal barrier function.

### SUMMARY

In point of fact, the applicant company has now found that an alcoholic extract of jasmine, Jasminum grandiflorum L., stems, obtained by a particular process, exhibits advantageous activities for counteracting the negative effects of pollution, in particular exhibits certain cosmetic properties in the strengthening of the cutaneous barrier, hydration, remodeling of the cutaneous matrix and protection against pollution. This is because, as demonstrated by examples, the alcoholic extract of jasmine, Jasminum grandiflorum L., stems according to the invention counteracts the negative effect of pollution by stimulating the targets involved in the barrier function of the skin, such as AQP1, CASP14, KRT1, KRT10, TGFb2 and FLG. In addition, the extract is capable of improving the remodeling of the matrix structure by stimulating COL4A1, COL8A1, DPT and P4HA1.

The invention thus relates, according to a first aspect, to an alcoholic extract of jasmine, Jasminum grandiflorum L., stems comprising at least oleuropein, isoquercitrin and ursolic acid.

Another subject matter of the invention, according to a second aspect, is a process for the extraction of jasmine, Jasminum grandiflorum L., stems comprising the following stages:
a) the grinding of the jasmine, Jasminum grandiflorum L., stems;
b) at least one, preferably at least two, extractions of said ground stems in the presence of a first alcoholic solvent, each extraction being followed by a filtration stage;
c) the mixing of the filtrates resulting from the extractions of stage b), then the filtration of said mixture;
d) the concentration of the mixture obtained in stage c), in order to obtain an extract having a dry matter content of between 8% and 13%, the remainder of the extract being composed of the first alcoholic solvent;
e) the addition of a second alcoholic solvent to the concentrated extract obtained in stage d), said second alcoholic solvent being different from the first alcoholic solvent used in stage b), followed by a removal of the first alcoholic solvent down to a final concentration of less than 0.5%;
f) the final dilution of the extract obtained in stage e) in an alcoholic solvent identical to the second alcoholic solvent used in stage e); and
g) optionally the filtration of the extract obtained in stage f).

The invention also relates to an alcoholic extract of jasmine, Jasminum grandiflorum L., stems capable of being obtained by such a process.

The invention additionally relates also to a cosmetic composition comprising, in a cosmetically acceptable vehicle, an alcoholic extract of jasmine, Jasminum grandiflorum L., stems according to the invention. The term “cosmetically acceptable vehicle” is understood to mean a medium compatible with the skin, the mucous membranes and the superficial body growths. Preferably, the cosmetic composition according to invention is suitable for application by the topical route.

### DETAILED DESCRIPTION

The alcoholic extract which is a subject matter of the invention results from jasmine, Jasminum grandiflorum L., stems, or vine shoots or branches. Preferably, dried jasmine stems are used. These dried jasmine stems are obtained from fresh stems which can be dehydrated either in the sun or under vacuum at a temperature ranging up to 60° C.

Jasminum grandiflorum L. is a species of the genus Jasminum of the family of the Oleaceae, known as Royal jasmine, Spanish jasmine, Catalan jasmine and Sicilian jasmine.

Jasminum grandiflorum exists as a shrub of approximately 2 to 4 m in height. The branches are green, glabrous and striated. The foliage is deciduous to semipermanent. Its flexible stems carry persistent leaves and large white flowers.

The inventors have concerned themselves with the jasmine stems obtained on conclusion of the harvesting of the flowers, during the annual pruning in November-December, necessary for the growth of the plant. This makes it possible in particular to upgrade the coproducts of the harvesting of the flowers.

The Jasminum grandiflorum L. dry extract which is a subject matter of the present invention is composed of several families of primary and secondary metabolites of interest.

The alcoholic extract of jasmine, Jasminum grandiflorum L., stems which is a subject matter of the present invention comprises in particular at least oleuropein, isoquercitrin and ursolic acid.

In particular, the alcoholic extract of jasmine, Jasminum grandiflorum L., stems of the invention comprises:
from 1% to 10% by weight of oleuropein, preferably from 5% to 7% by weight,
from 0.1% to 1% by weight of isoquercitrin, preferably from 0.3% to 0.5% by weight, and
from 0.1% to 1% by weight of ursolic acid, preferably from 0.5% to 0.7% by weight, the percentages being expressed by weight, with respect to the total weight of the dry extract.

Oleuropein is a compound corresponding to the following formula I:

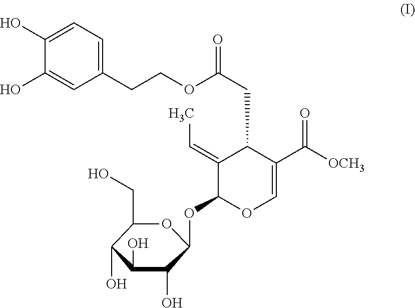

Isoquercitrin is a compound corresponding to the following formula II:

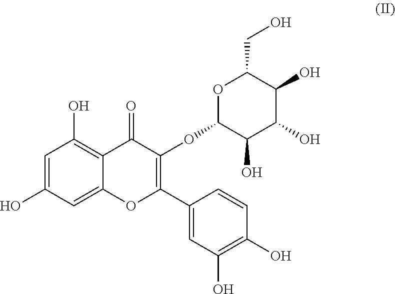 [SMILES: O=c1c(O[C@@H]2O[C@H](CO)[C@@H](O)[C@H](O)[C@H]2O)c(-c2ccc(O)c(O)c2)oc2cc(O)cc(O)c12]

Ursolic acid is a compound corresponding to the following formula III:

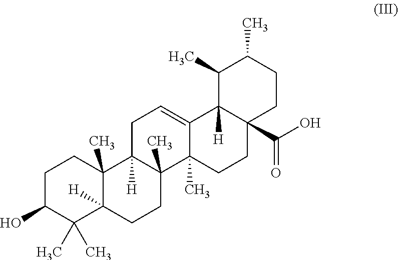

Preferably, said alcoholic extract of jasmine stems of the invention additionally comprises saccharides and/or mannitol, for example between 40% and 70% by weight of saccharides and/or of mannitol, preferably from 50% to 60% by weight, the percentages being expressed by weight, with respect to the total weight of the dry extract.

In particular, the alcoholic extract of jasmine, Jasminum grandiflorum L., stems of the invention comprises:
from 40% to 70% by weight of saccharides and/or of mannitol, preferably from 50% to 60% by weight,
from 1% to 10% by weight of oleuropein, preferably from 5% to 7% by weight,
from 0.1% to 1% by weight of isoquercitrin, preferably from 0.3% to 0.5% by weight, and
from 0.1% to 1% by weight of ursolic acid, preferably from 0.5% to 7% by weight, the percentages being expressed by weight, with respect to the total weight of the dry extract.

The saccharides present in the extract of jasmine stems according to the invention preferably comprise fructose, glucose and/or sucrose, and in particular from 2% to 5% by weight of fructose, from 20% to 25% by weight of glucose and/or from 2% to 5% by weight of sucrose, the percentages being expressed by weight, with respect to the total weight of the dry extract.

Preferably, the extract of jasmine stems according to the invention comprises from 20% to 25% by weight of mannitol, the percentages being expressed by weight, with respect to the total weight of the dry extract.

It is to the credit of the applicant company to have succeeded in preparing an extract of jasmine stems comprising at least oleuropein, isoquercitrin and ursolic acid, which makes its specificity therefrom.

The invention which is a subject matter of the present patent application also relates to a process for the extraction of jasmine, Jasminum grandiflorum L., stems comprising the following stages:
a) the grinding of the jasmine, Jasminum grandiflorum L., stems;
b) at least one, preferably at least two, extractions of said ground stems in the presence of a first alcoholic solvent, each extraction being followed by a filtration stage;
c) the mixing of the filtrates resulting from the extractions of stage b), then the filtration of said mixture;
d) the concentration of the mixture obtained in stage c), in order to obtain an extract having a dry matter content of between 8% and 13%, the remainder of the extract being composed of the first alcoholic solvent;
e) the addition of a second alcoholic solvent to the concentrated extract obtained in stage d), said second alcoholic solvent being different from the first alcoholic solvent used in stage b), followed by a removal of the first alcoholic solvent down to a final concentration of less than 0.5%;
f) the final dilution of the extract obtained in stage e) in an alcoholic solvent identical to the second alcoholic solvent used in stage e); and
g) optionally the filtration of the extract obtained in stage f).

Preferably, the jasmine stems employed in stage a) are dried stems. These dried stems are obtained from fresh stems which can be dehydrated either in the sun or under vacuum at a temperature ranging up to 60° C.

They are in particular ground or reduced to pieces, for example to a fineness of less than 2 cm, by any means known to a person skilled in the art.

In stage b), the ground jasmine stems are subjected to at least one, preferably at least two, extractions by one or more alcoholic solvents, i.e. first alcoholic solvent, which are chosen from C₁-C₄ monoalcohols, such as, for example, methanol, ethanol, propanol, isopropanol, butanol or isobutanol.

Preferably, the first alcoholic solvent of stage b) is a monoalcohol comprising from 2 to 4 carbon atoms, more preferentially ethanol.

In particular, the ethanol is employed as a 96% mixture with water.

The extraction is generally carried out by immersing and by gently stirring the jasmine stems in one or more of the abovementioned solvents for a period of time of between 1 and 6 hours, preferably between 2 and 4 hours, at a temperature of between 50° C. and 70° C., preferably between 55° C. and 65° C.

Preferably, the jasmine stems are extracted in ethanol with a 1/10 ratio by weight of jasmine stems with respect to the weight of ethanol.

The extraction stage b) is followed by a filtration, for example over a 100 μm filtration membrane, to remove the plant residues.

According to a preferred embodiment, stage b) employs at least two extractions. In this case, each extraction is followed by a filtration stage. Preferably, the first extraction is carried out over a period of time of 4 h at a temperature of 60° C. and the second extraction is carried over a period of time of 2 h at a temperature of 60° C.

On conclusion of the extraction stage or stages b), the liquid filtrates are recovered and mixed.

In stage c), the filtrate or the mixture filtrates resulting from the extractions of stage b) are then again filtered in order to remove the insoluble substances, preferably over a 15 μm filtration membrane. A liquid filtrate is thus obtained.

Preferably, between stages c) and d), a stage of decoloration of the mixture obtained in c) is added. The decoloration can be carried out by adsorption of the pigments present in the filtrate on activated carbon. Preferably, the activated carbon represents 40% by weight, with respect to the extract dry matter. This decoloration stage can be followed by a stage of filtration of the decolored filtrate obtained, preferably over a 4 μm filtration membrane, in order to remove the activated carbon charged.

In stage d), the mixture obtained in stage c) is concentrated in order to obtain an extract having a dry matter content of between 8% and 13%, the remainder of the extract being composed of the first alcoholic solvent. Preferably, the concentrated extract obtained in stage d) has a dry matter content of between 10% and 11%.

Preferably, the concentration of stage d) is carried out by evaporation, in particular by partial evaporation under reduced pressure, for example by means of a rotary evaporator or of a thin film evaporator.

In stage e), a second alcoholic solvent is subsequently added to the concentrated extract obtained in stage d), said second alcoholic solvent being different from the first alcoholic solvent used in stage b), followed by a removal of the first alcoholic solvent down to a final concentration of less than 0.5%.

The second alcoholic solvent of stage e) can be chosen from C₁-C₄ monoalcohols, such as, for example, methanol, ethanol, propanol, isopropanol, butanol or isobutanol; polyols, in particular diols, such as, for example, propylene glycol, 1,3-propanediol or dipropylene glycol, or triols, such as glycerol.

Preferably, the second alcoholic solvent of stage e) is a polyol comprising from 1 to 4 carbon atoms, more preferentially 1,3-propanediol.

More preferably still, the first alcoholic solvent of stage b) is a monoalcohol comprising from 2 to 4 carbon atoms, more preferentially ethanol, and the second alcoholic solvent of stages e) and f) is a polyol comprising from 1 to 4 carbon atoms, more preferentially 1,3-propanediol.

Preferably, the removal of the first alcoholic solvent of stage e) is carried out by evaporation, in particular by complete evaporation under reduced pressure, for example by means of a thin film evaporator.

In particular, in stage e), the second alcoholic solvent is added in proportions corresponding to between 5 and 7 times, preferably 6 times, the weight of the dry extract of the filtrate obtained in stage d).

Finally, in stage f), the extract obtained in stage e) is diluted in an alcoholic solvent identical to the second alcoholic solvent used in stage e) and defined above, preferably a polyol comprising from 1 to 4 carbon atoms, more preferentially 1,3-propanediol.

Preferably, the extract obtained in stage f) has a dry matter content of between 9.5% and 10.5%, preferably of 10%.

Optionally, in stage g), the extract obtained in stage f) is filtered, preferably over a 4 μm filtration membrane.

According to a particular form of the invention, there is added, between stages d) and e), a stage of storage and of cooling of the concentrated extract obtained in stage d), preferably at a temperature of 4° C. for at least 24 h, followed by a stage of filtration, preferably over a 40 μm filtration membrane, and/or,
(ii) between stages e) and f), there is added a stage of storage and of cooling of the extract obtained in stage e), preferably at a temperature of 4° C. for at least 24 h, followed by a stage of filtration, preferably over a 15 μm filtration membrane.

Preferentially, the alcoholic extract of jasmine, Jasminum grandiflorum L., stems according to the invention is capable of being obtained by the process described above comprising the following stages:
a) the grinding of the jasmine, Jasminum grandiflorum L., stems;
b) at least one, preferably at least two, extractions of said ground stems in the presence of a first alcoholic solvent, each extraction being followed by a filtration stage;
c) the mixing of the filtrates resulting from the extractions of stage b), then the filtration of said mixture;
d) the concentration of the mixture obtained in stage c), in order to obtain an extract having a dry matter content of between 8% and 13%, the remainder of the extract being composed of the first alcoholic solvent;
e) the addition of a second alcoholic solvent to the concentrated extract obtained in stage d), said second alcoholic solvent being different from the first alcoholic solvent used in stage b), followed by a removal of the first alcoholic solvent down to a final concentration of less than 0.5%;
f) the final dilution of the extract obtained in stage e) in an alcoholic solvent identical to the second alcoholic solvent used in stage e); and
g) optionally the filtration of the extract obtained in stage f).

Advantageously, the extract employed according to the invention is light in color.

Equally, said extract is provided in a sufficiently concentrated form to be able to be used without causing the formulation problems normally encountered at the concentrations necessary in order to obtain an activity in cosmetic or dermatological compositions in the emulsion form, and without exhibiting a dark color, unlike the plant extracts obtained by normal processes, when they are in concentrated form.

In particular, after evaporation of the ethanol, the extract of jasmine, Jasminum grandiflorum L., stems comprises from 5% to 15% by weight of jasmine dry extract and from 85% to 95% by weight of 1,3-propanediol, and preferably approximately 10% by weight of jasmine dry extract and 90% by weight of 1,3-propanediol.

For this reason, the extract according to the invention can be used directly in the preparation of a cosmetic composition.

According to another aspect, the invention relates to the cosmetic use of an alcoholic extract of jasmine, Jasminum grandiflorum L., stems according to the invention to protect the skin against the harmful effects of pollution, for example against a mixture of pollutants composed of cigarette smoke and urban dust extracts.

According to yet another aspect, the invention relates to the cosmetic use of an alcoholic extract of jasmine, Jasminum grandiflorum L., stems according to the invention to improve the remodeling of the matrix structure.

According to yet another aspect, the invention relates to the cosmetic use of an alcoholic extract of jasmine, Jasminum grandiflorum L., stems according to the invention to improve the cutaneous barrier function and/or the hydration of the skin.

This is because, advantageously, it has been found that the alcoholic extract of jasmine, Jasminum grandiflorum L., stems according to the invention counteracted the negative effect of pollution by stimulating the targets involved in the barrier function of the skin, such as AQP1, CASP14, KRT1, KRT10, TGFb2 and FLG.

It has in particular been found, advantageously, that the alcoholic extract of jasmine, Jasminum grandiflorum L., stems according to the invention made it possible to improve the remodeling of the matrix structure by stimulating COL4A1, COL8A1, DPT and P4HA1.

The invention also relates, according to yet another aspect, to a cosmetic composition comprising, in a cosmetically acceptable vehicle, an alcoholic extract of jasmine, Jasminum grandiflorum L., stems according to the invention. Preferably, said extract is present in the cosmetic or dermatological composition in a proportion of from 0.001% to 10% by weight, with respect to the total weight of the composition, in particular in a proportion of from 0.01% to 10% by weight, preferably from 0.1% to 10% by weight. Said cosmetic composition can in particular be suitable for application by the topical route.

Advantageously, said cosmetic composition can be provided in the form of a powder, of an emulsion, of a microemulsion, of a nanoemulsion, of a suspension, of a solution, of a lotion, of a cream, of an aqueous or aqueous/alcoholic gel, of a foam, of a serum, of a solution or of a dispersion for an aerosol, or of a dispersion of lipid vesicles.

In the case of an emulsion, it can be a water-in-oil or oil-in-water emulsion.

The cosmetic composition according to the invention can be provided in the form of composition for caring for and/or making up keratin materials, preferably of the face.

In particular, the composition of the invention can be provided in the form of an antiaging care composition for the skin, in particular of the face.

According to another embodiment, the composition of the invention can be provided in the form of a composition for making up the face, in particular in the form of a foundation, of a complexion base or of a tinted cream.

The cosmetic composition according to the invention can also comprise a solvent chosen as a function of the various ingredients and of the form of administration.

Mention may be made, by way of examples, of water (preferably demineralized water or floral waters) or alcohol, such as ethanol.

Said cosmetic composition can also comprise, besides the extract according to the invention, at least one additive normal in the field, such as, for example, at least one compound chosen from an emollient or humectant agent, a gelling and/or thickening agent, a surface-active agent, an oil, an active agent, a colorant, a preservative, an antioxidant, an active agent, an organic or inorganic powder, a sunscreen and a fragrance.
One or more humectant agent(s), such as polyols (glycerol, diglycerol, propylene glycol, propanediol, caprylyl glycol, pentylene glycol, hexanediol), sugars, glycosaminoglycans, such as hyaluronic acid and its salts and esters, and polyquaterniums.

Said humectant agent will be present in the composition at a content of the order of from 0.1% to 30%, preferably from 0.005% to 10%, by total weight of the composition.
One or more emollient agent(s) which can be chosen, for example, from esters, such as jojoba esters, esters of fatty acids and of fatty alcohol (octyldodecyl myristate, triethylhexanoin, dicaprylyl carbonate, isostearyl isostearate, caprylic/capric triglyceride), butters, such as shea butters (Butyrospermum parkii butter extract, shea butter ethyl esters) or moringa butters (moringa oil/hydrogenated moringa oil esters), waxes (Acacia decurrens flower wax & Helianthus annuus cera seed wax, C10-18 triglycerides), vegetable oils, phytosqualane or alkanes (undecane, tridecane).

Said emollient agent will be present in the composition at a content of the order of from 0.1% to 30%, preferably from 0.5% to 10%, by total weight of the composition.
One or more gelling and/or thickening agent(s) for the aqueous phase, for example chosen from cellulose derivatives, gums of vegetable origin (guar, locust bean, alginates, carrageenans, pectins), of microbial origin (xanthan), clays (laponite), homo- and copolymers which are crosslinked or noncrosslinked and hydrophilic or amphiphilic, of acryloylmethylpropane sulfonic acid (AMPS) and/or of acrylamide and/or of acrylic acid and/or of acrylic acid salts or esters (such as Ammonium AMP/VP Copolymer, Hydroxyethyl Acrylate/AMPS Copolymer, Sodium Acrylate/AMPS Copolymer, Acrylamide/AMPS Copolymer, Acrylates Copolymer, Acrylates/C10-30 Alkyl Acrylate Crosspolymer, Polyacrylate Crosspolymer-6, Sodium Polyacrylate).

Said gelling and/or thickening agent will be present in the composition at a content of the order of from 0.1% to 10% by total weight of the composition.
One or more surface-active agent(s), in particular:
anionic surfactants, such as isethionates, taurates, sarcosinates, glycinates, glutamates, phosphates, such as C20-22 alkyl phosphate,
amphoteric surfactants, such as betaine derivatives or amphoacetates,
nonionic surfactants, such as polyglycerol derivatives, derivatives of glucosides (such as cetearyl glucoside, C12-20 alkyl glucoside, arachidyl glucoside, caprylyl/capryl glucoside, decyl glucoside, lauryl glucoside), xyloside derivatives, lecithins.

Said surface-active agent will be present in a content of the order of from 0.1% to 15%, preferably from 0.5% to 10%, by weight, with respect to the total weight of the composition.
One or more film-forming agent(s), such as polymers of natural origin, which are optionally modified, can be chosen from shellac resin, gum sandarac, gum arabic (Acacia Senegal Gum), dammars, elemis, copals, cellulose polymers, polymers extracted from the fruit of Caesalpinia spinosa and/or from the alga Kappaphycus alvarezii, and their mixtures.

Said film-forming agent will be present in a content of the order of from 0.1% to 15%, preferably from 0.5% to 10%, by weight, with respect to the total weight of the composition.
One or more coloring agent(s) chosen from pigments, pearlescent agents or soluble dyes, preferably water-soluble dyes. The pigments can be white or colored and inorganic and/or organic. The inorganic pigments can be chosen from zirconium or cerium oxides, and also zinc, iron or chromium oxides or titanium dioxide. The pearlescent agents can be chosen from pearlescent pigments, such as titanium oxide-coated mica covered with an iron oxide, titanium oxide-coated mica covered with bismuth oxychloride, titanium oxide-coated mica covered with chromium oxide or titanium oxide-coated mica covered with an organic dye, and also pearlescent pigments based on bismuth oxychloride.

Said coloring agent will be present in a content of the order of from 0.01% to 20%, preferably from 2% to 15%, by weight, with respect to the total weight of the composition.
One or more filling agent(s) chosen from lauroyl lysine, starch, boron nitride and silica.

Said filling agent will be present in a content of the order of from 0.1% to 10%, preferably from 0.5% to 7%, by weight, with respect to the total weight of the composition.

One or more active agent(s) of natural, biotechnological or synthetic origin having a biological activity and having an effectiveness on the skin via biological sites, for example chosen from vitamins, such as vitamin C and its derivatives (ascorbyl glucoside, 3-O-ethyl ascorbic acid, ascorbyl tetraisopalmitate), vitamin A and its derivatives, vitamin E and its derivatives, vitamin B3 or niacinamide, panthenol, trace elements, allantoin, adenosine, peptides (palmitoyl tetrapeptide-7, palmitoyl tripeptide-1, palmitoyl pentapeptide-4, acetyl dipeptide-1 cetyl ester, acetyl tetrapeptide-5), plant extracts (Glycyrrhiza glabra extract, Centella asiatica leaf extract, Secale cereale seed extract), extracts of yeasts, α-hydroxy acids, such as glycolic or lactic acid, tranexamic acid and its derivatives, such as cetyl tranexamic acid, and the like.

Said active agent will be present in the composition at a content of the order of from 0.1% to 10% by total weight of the composition.

Other additives normally used in cosmetics can also be present in the composition according to the invention, in particular preservatives, antioxidants and fragrances well known in the technical field.

A person skilled in the art is in a position to choose, from all of these possible additives, both the nature and the amount of those which will be added to the composition, so that the latter retains all of its properties.

The invention is illustrated without limitation by the examples below.

### Example 1: Preparation of an Alcoholic Extract of Jasmine, Jasminum grandiflorum L., Stems According to the Invention

An alcoholic extract of jasmine, Jasminum grandiflorum L., stems according to the invention was prepared by a process comprising the following stages: 1 kg of fresh branches were dried by dehydrating them either in the sun or under vacuum at a temperature ranging up to 60° C. and then were subsequently coarsely ground. The branches were extracted in 10 kg of 96° ethanol with stirring at 60° C. for 4 h;
The mixture was sieved at 100 μm in order to recover the spent material and the extract was set aside;
The spent material was again extracted in 8 kg of 96° ethanol with stirring at 60° C. for 2 h;
The spent crushed branches were removed by solid/liquid separation and the liquid fractions were combined and then filtered at 15 μm;
The ethanolic fraction was decolored with 140 g of activated carbon (40% by weight with respect to the extract dry matter) with stirring at ambient temperature for 3 h;
The activated carbon was subsequently removed by filtration at 4 μm;
The ethanolic extract was concentrated by evaporation of the ethanol on a rotary evaporator or on a falling film evaporator in order to obtain a concentrated extract with a dry matter of between 10% and 11%;
The combined product was stored at 4° C. for several days;
The solid phase was removed by filtration at 40 μm;
The measurement of dry matter was carried out on the extract and contained 61 g of dry extract;
6 times the weight of extract of propanediol, i.e. 368 g, were added. The ethanol was completely evaporated on a thin film evaporator (final ethanol concentration less than 0.5%);
The extract was left at 4° C. overnight and filtered at 15 μm;
367 g of propanediol were added, so that the dry extract is at a concentration of 10%;
A final filtration at 4 μm was carried out;
780 g of final extract were obtained.

### Example 2: Modulations of the Expression of the Genes Involved in the Cutaneous Barrier Function and the Remodeling of the Matrix in Skin Explants Stressed by a Mixture of Pollutants and Treated with an Extract of Jasmine Stems

Protocol: The skin explants of 3 different donors were supplied on day 0 and two technical replicas for each condition were produced. A test with alamarBlue was carried out to test the cytotoxicity of the jasmine stem extract. Consequently, an amount of 50 μl at a noncytotoxic 0.01% or 0.05% concentration of the final extract obtained in example 1 was added to the top of the skin for 1 hour, and a cotton swab (with parafilm) was used to gently massage the explant. Under the control conditions, PBS (phosphate buffer saline) was used and applied in the same way to the skin explants. In addition, the extract was also introduced into the culture medium (systemic application) at the concentrations already mentioned. After 1 hour, the overflow was removed. The same protocol was carried out on day 3 (replacement with a fresh extract) where a mixture of pollutants, composed of cigarette smoke and urban dust extracts, was used to induce stress for 6 h. At the end of the stress period (6 h), each skin explant was withdrawn from the insert and placed in the lysis tube in order to extract the RNAs using the QIAzol and GeneJEt RNA purification kit according to the supplier's instructions. Real-time reverse transcription and PCR were subsequently carried out in in order to quantify the modulation of the expression of the genes. The level of expression of the mRNAs was standardized with respect to the following reference genes: YWHAZ, GAPDH, HPRT, β-Actin and RPL13. The results are expressed as induction threshold with respect to the PBS control condition and to the condition of pollution mixture without treatment with the jasmine stem extract (stress control) and are obtained by using the AACt method, where the Ct correspond to the number of cycles necessary to generate a fluorescent signal above the predefined threshold (the more targets there are, the fewer cycles are necessary to reach this threshold). The main interest concerned the genes of the category Ct<20.

Results: The application of a mixture of pollutants (control of the stress) to normal skin showed a decrease in the expression of the genes involved in various cutaneous mechanisms, such as the barrier function, or the remodeling of the matrix in comparison with the PBS control condition (Table II). The jasmine stem extract counteracted the negative effect of the pollution by stimulating the targets involved in the barrier function of the skin, such as AQP1, CASP14, KRT1, KRT10, TGFb2 and FLG, and also the targets involved in the remodeling of the matrix structure by stimulating COL4A1, COL8A1, DPT and P4HA1. The induction thresholds for the expression of the genes are defined in Table I. The functional definition and the classification of the modulated genes mentioned above are described in Table III.

TABLE I
Induction threshold for the expression of the genes.

| | | |
|---|---|---|
| 1 to 4 | x1 to x4 | Stimulation of the |
| | | expression of the genes |
| 0.8 to 1.2 | 0.8x to 1.2x | Modulation of the |
| | | expression of the genes not |
| | | significant |
| 0.8 to 0.65 | /1.2 to /2 | Inhibition of the expression |
| | | of the genes |

TABLE II
List of the genetic expressions modulated by the mixture of pollutants at 6 h
after the stress, with or without treatment with the extract of jasmine stems.

| | | | | | Level of | |
|---|---|---|---|---|---|---|
| | | Level of expression compared | | | expression compared with | |
| | | with the PBS control conditions | | | the control conditions of | |
| | | Don1 + | Don2 + | Don3 + | the mixture of pollutants | |
| | Name of | mixture of | mixture of | mixture of | +0.05% | +0.01% |
| Symbol | the genes | pollutants | pollutants | pollutants | extract | extract |
| AQP1 | aquaporin 1 | 0.79 | 0.50 | 0.92 | 1.66 | 2.15 |
| CASP14 | caspase 14 | 0.67 | 0.78 | 0.76 | 1.27 | 1.47 |
| COL4A1 | collagen type | 0.84 | 0.59 | 0.96 | 1.37 | 1.23 |
| | IV alpha 1 | | | | | |
| COL8A1 | collagen type | 0.77 | 0.94 | 0.88 | 1.68 | 1.76 |
| | VIII alpha 1 | | | | | |
| | chain | | | | | |
| DPT | dermatopontin | 0.70 | 0.71 | 0.93 | 1.29 | 1.13 |
| FLG | filaggrin | 0.60 | 0.68 | 0.80 | 1.54 | 1.79 |
| KRT1 | keratin 1 | 0.73 | 0.78 | 0.98 | 2.46 | 2.16 |
| KRT10 | keratin 10 | 0.76 | 0.82 | 0.96 | 2.87 | 2.58 |
| P4HA1 | prolyl 4- | 0.71 | 0.62 | 0.88 | 1.40 | 1.47 |
| | hydroxylase | | | | | |
| | subunit alpha | | | | | |
| | 1 | | | | | |
| TGFb2 | transforming | 0.79 | 1.05 | 0.74 | 2.59 | 2.75 |
| | growth factor | | | | | |
| | beta2 | | | | | |

TABLE III
Functional definition and classification of the modulated genes.

| | | | FUNCTIONAL |
|---|---|---|---|
| GENES | NAME | FUNCTION | CLASSIFICATION |
| AQP1 | Aquaporin 1 | Transmembrane | Hydration/Barrier |
| | | proteins involved | Function |
| | | in the | |
| | | transportation of | |
| | | water | |
| CASP14 | Caspase 14 | Enzyme involved | Barrier Function |
| | | in the cleavage of | |
| | | profilaggrin to | |
| | | give filaggrin | |
| FLG | Filaggrin | Major structural | Barrier Function |
| | | protein in terminal | |
| | | differentiation and | |
| | | the formation of | |
| | | horny envelopes in | |
| | | the horny layer | |
| KRT1 | Keratin 1 | Differentiation of | Barrier Function |
| | | simple and | |
| | | stratified epithelial | |
| | | tissues | |
| KRT10 | Keratin 10 | Proteins which | Barrier Function |
| | | form the | |
| | | intermediate | |
| | | filament | |
| | | (cytoskeleton of | |
| | | epithelial cells) | |
| TGFb2 | Transforming | Cytokine involved | Barrier Function |
| | growth factor | in cell | |
| | beta2 | differentiation and | |
| | | proliferation | |
| COL4A1 | Collagen type IV | Protein of the | Structure |
| | alpha 1 | basement | |
| | | membrane | |
| COL8A1 | Collagen type | Protein of the | Structure |
| | VIII alpha 1 | basement | |
| | chain | membrane | |
| DPT | Dermatopontin | Protein of the | Structure |
| | | extracellular | |
| | | matrix involved in | |
| | | cell-matrix | |
| | | interactions and | |
| | | the assembling of | |
| | | the matrix | |
| P4HA1 | Prolyl 4- | Key enzyme in | Structure |
| | hydroxylase | the synthesis | |
| | subunit alpha 1 | of collagen | |

Conclusion: By virtue of its ability to counteract the negative effects of the pollution, as is shown in Table II, the extract of jasmine stems exhibits certain cosmetic properties in the strengthening of the cutaneous barrier, hydration, remodeling of the cutaneous matrix and protection against pollution.

### Example 3: Cosmetic Compositions

The following compositions can be prepared in a way conventional to a person skilled in the art. The amounts shown below are expressed as percentages by weight. The ingredients in capital letters are identified in accordance with the INCI designation.

### A—Fluid Oil/Water Emulsion

| INCI name | (% w/w) |
|---|---|
| Limnanthes alba (meadowfoam) seed oil | 1-10 |
| Camellia oleifera seed oil | 1-10 |
| Caprylic/capric triglyceride | 1-10 |
| Dicaprylyl dicarbonate | 1-5 |
| Squalane | 1-10 |
| Ammonium acryloyldimethyltaurate/VP | 0.1-5 |
| copolymer | |
| Hydroxyethyl acrylate/sodium | 0.1-2 |
| acryloyldimethyltaurate copolymer & | |
| sorbitan isostearate & polysorbate 60 | |
| Xanthan gum | 0.01-5 |
| Biosaccharide gum-1 | 0.01-5 |
| Sodium hyaluronate | 0.01-3 |
| Glycerin | 1-30 |
| Polyquaternium-51 | 1-10 |
| Tocopheryl acetate | 0.1-5 |
| Niacinamide | 0.1-5 |
| Polianthes tuberosa | 0.01-5 |
| Sodium PCA | 0.5-5 |
| Saccharide isomerate | 0.5-5 |
| Extract of jasmine stems according to the | 0.001-10 |
| invention | |
| Yeast extract | 0.1-5 |
| CI 77891 (titanium dioxide) & aluminum | 1-10 |
| hydroxide & magnesium chloride & | |
| sodium lauroyl glutamate & lysine | |
| CI 77891 (titanium dioxide) & mica & tin | 1-10 |
| oxide | |
| Kappaphycus alvarezii extract & | 0.1-5 |
| Caesalpinia spinosa fruit extract | |
| Titanium dioxide & stearic acid & alumina | 1-7 |
| Phenylbenzimidazole sulfonic acid | 1-3 |
| Glycols (caprylyl glycol and/or pentylene | 0.1-10 |
| glycol and/or butylene glycol and/or | |
| propanediol) | |
| Water | q.s. for 100 |

### B—Cream Oil/Water Emulsion

| INCI name | (% w/w) |
|---|---|
| Jojoba esters | 1-5 |
| Limnanthes alba (meadowfoam) seed oil | 0.1-5 |
| Canola oil | 1-10 |
| Argania spinosa kernel oil | 0.1-10 |
| Moringa oil/hydrogenated moringa oil | 1-10 |
| esters | |
| C8-12 acid triglyceride | 1-5 |
| Lauroyl lysine | 1-5 |
| Camellia oleifera seed oil | 1-10 |
| Squalane | 1-10 |
| Ammonium acryloyldimethyltaurate/VP | 1-5 |
| copolymer | |
| Cetearyl alcohol & cetearyl glucoside | 1-7 |
| Sodium lauroyl glutamate | 0.1-1 |
| Hydrogenated lecithin | 0.1-5 |
| Chondrus crispus (carrageenan) | 0.1-5 |
| Sclerotium gum | 0.01-2 |
| Centella asiatica leaf extract | 0.1-5 |
| Adenosine | 0.1-0.5 |
| Niacinamide | 0.1-5 |
| Secale cereale (rye) seed extract | 0.1-5 |
| Palmitoyl tripeptide-1 & palmitoyl | 1-5 |
| tetrapeptide-7 | |
| Plankton extract | 0.1-5 |
| Yeast extract | 1-3 |
| Extract of jasmine stems according to | 0.001-10 |
| the invention | |
| Glycyrrhiza glabra extract | 0.001-5 |
| Tranexamic cetyl ester | 0.001-5 |
| Ascorbyl glucoside | 0.001-5 |
| Water | q.s. for 100 |

These compositions can be applied every day, morning and/or evening, to the skin.

### Example 4: Composition of an Alcoholic Extract of Jasmine, Jasminum grandiflorum L., Stems According to the Invention

The alcoholic extract of jasmine stems according to the invention comprises diverse families of primary and secondary metabolites. Among the primary metabolites, glucose, fructose, sucrose and mannitol have been identified. Among the secondary metabolites, oleuropein, isoquercitrin and ursolic acid have been identified (Table IV).

TABLE IV
Composition of a final extract and of a dry extract of jasmine,
Jasminum grandiflorum L., as obtained in example 1

| Family | Molecule | (% final extract) | (% dry extract) |
|---|---|---|---|
| Secoiridoids | Oleuropein | 0.6 | 6.7 |
| | (C₂₅H₃₂O₁₃) | | |
| Flavonoids | Isoquercitrin | 0.004 | 0.4 |
| Pentacyclic | Ursolic acid | 0.1 | 0.6 |
| triterpenoids | (C₃₀H₄₈O₃) | | |
| Sugars/Polyols | Fructose | 0.3 | 3.3 |
| | Glucose | 2.2 | 21.9 |
| | Sucrose | 0.3 | 2.7 |
| | Mannitol | 2.2 | 22.4 |
| Dilution solvent | 1,3-Propanediol | 90.0 | / |
| Total | | 95.7 | 58.0 |

1. An alcoholic extract of jasmine, Jasminum grandiflorum L., stems comprising at least oleuropein, isoquercitrin and ursolic acid.

2. The alcoholic extract of jasmine stems as claimed in claim 1, comprising:
from 40% to 70% by weight of saccharides and/or of mannitol, preferably from 50% to 60% by weight,
from 1% to 10% by weight of oleuropein, preferably from 5% to 7% by weight,
from 0.1% to 1% by weight of isoquercitrin, preferably from 0.3% to 0.5% by weight, and
from 0.1% to 1% by weight of ursolic acid, preferably from 0.5% to 0.7% by weight,
the percentages being expressed by weight, with respect to the total weight of the dry extract.

3. The alcoholic extract of jasmine stems as claimed in claim 1 or 2, in which the saccharides comprise fructose, glucose and/or sucrose, preferably from 2% to 5% by weight of fructose, from 20% to 25% by weight of glucose and/or from 2% to 5% by weight of sucrose, and/or comprising from 20% to 25% by weight of mannitol, the percentages being expressed by weight, with respect to the total weight of the dry extract.

4. A process for the extraction of jasmine, Jasminum grandiflorum L., stems comprising the following stages:
a) the grinding of the jasmine, Jasminum grandiflorum L., stems;
b) at least one, preferably at least two, extractions of said ground stems in the presence of a first alcoholic solvent, each extraction being followed by a filtration stage;
c) the mixing of the filtrates resulting from the extractions of stage b), then the filtration of said mixture;
d) the concentration of the mixture obtained in stage c), in order to obtain an extract having a dry matter content of between 8% and 13%, the remainder of the extract being composed of the first alcoholic solvent;
e) the addition of a second alcoholic solvent to the concentrated extract obtained in stage d), said second alcoholic solvent being different from the first alcoholic solvent used in stage b), followed by a removal of the first alcoholic solvent down to a final concentration of less than 0.5%;
f) the final dilution of the extract obtained in stage e) in an alcoholic solvent identical to the second alcoholic solvent used in stage e); and
g) optionally the filtration of the extract obtained in stage f).

5. The process as claimed in claim 4, characterized in that the extractions of stage b) are carried out for a period of time of between 1 and 6 hours, preferably between 2 and 4 hours, at a temperature of between 50° C. and 70° C., preferably between 55° C. and 65° C.

6. The process as claimed in claim 4 or 5, characterized in that the first alcoholic solvent of stage b) is a monoalcohol comprising from 1 to 4 carbon atoms, preferably ethanol, and the second alcoholic solvent of stages e) and f) is a polyol comprising from 1 to 4 carbon atoms, preferably 1,3-propanediol.

7. The process as claimed in any one of claims 4 to 6, characterized in that, between stages c) and d), a stage of decoloration of the mixture obtained in c), preferably by adsorption on activated carbon, is added, followed by a stage of filtration of the decolored filtrate obtained, preferably over a 4 μm filtration membrane.

8. The process as claimed in any one of claims 4 to 7, characterized in that the concentration of stage d) is carried out by evaporation, in particular by partial evaporation under reduced pressure, and/or the removal of the first alcoholic solvent of stage e) is carried out by evaporation, in particular by complete evaporation under reduced pressure.

9. The process as claimed in any one of claims 4 to 8, characterized in that, in stage e), the second alcoholic solvent is added in proportions corresponding to between 5 and 7 times, preferably 6 times, the weight of the dry extract of the filtrate obtained in stage d).

10. The process as claimed in any one of claims 4 to 9, characterized in that:
(i) between stages d) and e), there is added a stage of storage and of cooling of the concentrated extract obtained in stage d), preferably at a temperature of 4° C. for at least 24 h, followed by a stage of filtration, preferably over a 40 μm filtration membrane, and/or
(ii) between stages e) and f), there is added a stage of storage and of cooling of the extract obtained in stage e), preferably at a temperature of 4° C. for at least 24 h, followed by a stage of filtration, preferably over a 15 μm filtration membrane.

11. The process as claimed in any one of claims 4 to 10, characterized in that:
(i) each extraction of stage b) is followed by a filtration over a 100 μm filtration membrane, and/or
(ii) the filtration carried out in stage c) is carried out over a 15 μm filtration membrane, and/or
(iii) the filtration carried out in stage g) is carried out over a 4 μm filtration membrane.

12. The process as claimed in any one of claims 4 to 11, characterized in that:
(i) the concentrated extract obtained in stage d) has a dry matter content of between 10% and 11%, and/or
(ii) the extract obtained in stage f) has a dry matter content of between 9.5% and 10.5%, preferably of 10%.

13. An alcoholic extract of jasmine, Jasminum grandiflorum L., stems, characterized in that it is capable of being obtained by a process as claimed in any one of claims 4 to 12.

14. The cosmetic use of an alcoholic extract of jasmine, Jasminum grandiflorum L., stems as claimed in any one of claims 1 to 3, for protecting the skin against the harmful effects of pollution, and/or improving the cutaneous barrier function and the hydration of the skin.

15. A cosmetic composition comprising, in a cosmetically acceptable vehicle, an alcoholic extract of jasmine, Jasminum grandiflorum L., stems as claimed in any one of claims 1 to 3.
